(19) United States
(12) Patent Application Publication
(10) Pub. No.: US 2024/0315188 A1
(43) Pub. Date: Sep. 26, 2024

(54) PROCESS FOR CONVERTING BIOMASS FROM PLANT CELL CULTURE INTO THREE-DIMENSIONAL STRUCTURE

(71) Applicant: TEKNOLOGIAN TUTKIMUSKESKUS VTT OY, Espoo (FI)

(72) Inventors: Heiko RISCHER, Espoo (FI); Elviira KÄRKKÄINEN, Espoo (FI); Christiane LAINE, Espoo (FI)

(21) Appl. No.: 18/575,564

(22) PCT Filed: Jul. 6, 2022
(86) PCT No.: PCT/FI2022/050492

§ 371 Date: Dec. 29, 2023

(30) Foreign Application Priority Data
Jul. 7, 2021 (FI) 20215796

(51) Int. Cl. A01H 4/00; C12N 5/00; C12N 5/04

(52) U.S. Cl. CPC A01H 4/005; C12N 5/0025; C12N 5/04

(57) ABSTRACT

The present disclosure relates to cellular agriculture and biotechnology-based methods to produce food. The disclosure especially relates to a process for converting biomass derived from a plant cell suspension culture into a three-dimensional plant biomass structure. The present disclosure further concerns a three-dimensional plant biomass structure obtained by the present method. The present disclosure further concerns a food product comprising said three-dimensional plant biomass structure.

### FIELD OF THE DISCLOSURE

The present disclosure relates to cellular agriculture and biotechnology-based methods to produce food. The disclosure especially relates to a process for converting biomass derived from a plant cell suspension culture into a three-dimensional plant biomass structure. The present disclosure further concerns a three-dimensional plant biomass structure obtained by the present method. The present disclosure further concerns a food product comprising said three-dimensional plant biomass structure.

### BACKGROUND OF THE DISCLOSURE

One of the major challenges of our time is turning the global food production systems towards healthy and environmentally sustainable diets. Plant cell cultures have been identified as a sustainable future food source containing valuable nutrients, health promoting substances and good sensory attributes. Increasing plant cell-based food sources provides improved health and environmental benefits.

Cellular agriculture refers to contained cultivation of cells to produce agricultural commodities. It is a new approach for plant-based food production through plant cell culture technology. Plant cell culture technology is a new approach, in the field of cellular agriculture, aiming to generate novel food solutions via biotechnology. Plant cells can offer an industrially scalable solution for the safe and controlled production of nutritional food raw material (Nordlund et al. 2018; Häkkinen et al. 2020). However, the loose and coarse appearance of cultivated plant cells has been one of the challenges related to this new technology.

Plant cell culture technology is based on the concept of cellular totipotency, the ability of cells to differentiate into a new organism. Mass propagation of de-differentiated plant cells has been studied for several decades. Pharmaceutical and cosmetic industries have utilized plant cells for specialized metabolite production.

Plant cell suspension cultures are widely used in plant biology as a convenient tool, bypassing the structural complexity of the plant organism in toto. The benefits of using suspension-cultured cells are homogeneity of an in vitro cell population, the large availability of material, the high rate of cell growth and the good reproducibility of conditions. Plant cell suspensions are cultivated in conventional stirred bioreactors, as individual cells or in small cell aggregates, and commercial cultivations up to 75,000 L prove their scaling up potential. Plant cell cultures can provide nutritiously superior ingredients without having the environmental challenges and uncontrollable quality variations of the conventional agriculture.

Plant cell biomass consisting of cells and cell aggregates have a gritty texture and result in sandy mouth feeling due to lack of tissue-like structure. Only very few studies have focused on structuring and processing biomass derived from cell cultures.

A patent publication WO 2010/059725 describes edible polymer hydrogel which was obtained by reacting CM-cellulose sodium salt and hydroxyethyl cellulose with citric acid in water.

Three-dimensional cellulose-based aerogels have been produced.

Despite the advances in the technology to utilize plant cell cultures in food production there remains a need of improved methods and products.

### BRIEF DESCRIPTION OF THE DISCLOSURE

An object of the present disclosure is to provide a process and product which overcome the above problems related to the conventionally used methods for producing food in plant cell cultures.

The object of the disclosure is achieved by a method and product which are characterized by what is stated in the independent claims. The preferred embodiments of the disclosure are disclosed in the dependent claims. The disclosure is based on the idea of converting biomass derived from a plant cell suspension culture into a three-dimensional plant biomass structure.

The present inventors surprisingly found that a three-dimensional plant biomass structure can be produced using a process described in the present disclosure. An advantage of the present process is that raw material is converted into an appealing three-dimensional form keeping or even improving its odour and/or changing or preserving the colour. It was surprisingly observed that the colour of the starting material can be maintained or preserved, when the right or optional conditions are used. In certain conditions, for example at high pH, and/or at high incubation temperature, the colour or visual appearance of the starting material may be changed. The present disclosure provides a process to structure a plant cell culture derived biomass broth in a simple and food grade way while preserving nutritional value, colour and flavour.

Another advantage of this process is that plant material can be produced sustainably, and hybrid products, e.g., mixtures of ingredients made biotechnologically by different hosts such as plant cells originating from different species, or combinations of plant and microbial cell mass, or combinations of plant cells and conventional food ingredients such as flour, can be produced.

In the present process plant cell biomass is recovered from the cultures and crosslinked. The main components of the plant cell wall cellulose, hemicellulose and pectin are crosslinked with citric acid. Citric acid is safe for food products.

The present disclosure provides a novel food structuring technology, offering a solution for the construction of edible structures from loose cell biomass produced in bioreactors. The ingredients used for structuring the cells are inexpensive and commonly used in the food industry. Thus, novel, nutritious food structures can be produced.

Cellular agriculture of plant cells is faster than culture of meat.

The disclosure relates to a process for converting biomass derived from a plant cell suspension culture into a three-dimensional plant biomass structure. The disclosure also relates to a three-dimensional plant biomass structure obtained by the present process. The disclosure also relates to a food product comprising the three-dimensional plant biomass structure and to the use of a three-dimensional plant biomass structure as an ingredient in a food product.

### BRIEF DESCRIPTION OF THE DRAWINGS

In the following the disclosure will be described in greater detail by means of preferred embodiments with reference to the accompanying drawings, in which

FIG. 1 illustrates filtered plant cells derived from plant cell suspension cultures. Unstructured plant cell biomass is shown. In upper row from left to right: rowan, lingonberry, arctic bramble. In lower row from left to right: strawberry, cloudberry, raspberry.

FIG. 2 illustrates a three-dimensional plant biomass structure, i.e., plant cell patty, i.e., foamy structured plant cells, which form three-dimensional plant biomass structure. The plant cell patty is formed of a mixture of strawberry cells and arctic bramble cells.

FIG. 3 illustrates film-like structured rowan cells. Two different samples are shown.

FIG. 4A illustrates average particle size distribution of rowan cell suspension cultures. Volume density (%) and particle size (μm) are presented.

FIG. 4B illustrates average particle size distribution of arctic bramble cell suspension cultures. Volume density (%) and particle size (μm) are presented.

FIG. 4C illustrates light microscopy image of rowan cells. Scale bar=100 μm.

FIG. 4D illustrates light microscopy image of arctic bramble cells. Scale bar=100 μm.

FIG. 5A illustrates an XRT image of rowan, i.e., a microstructure of rowan cell patty characterized by XμCT.

FIG. 5B illustrates an XRT image of arctic bramble, i.e., a microstructure of arctic bramble patty characterized by XμCT.

FIG. 6 illustrates porosity (%) of rowan cell patties and arctic bramble cell patties measured in XRT analysis. Treatments from left to right: control, low NaOH, high NaOH, low temperature, high temperature, no incubation, long incubation, low NH₄OH, high NH₄OH, low CA & sorbitol, high CA & sorbitol. Letters a, b, c, and d represent statistical differences between these groups with the confidence level p<0.05 (Tukey HSD).

FIG. 7 illustrates mechanical properties of plant cell patties. Toughness (kg*mm) of rowan cell patties and arctic bramble cell patties with different treatments measured in compression test is presented. Treatments from left to right: control, low NaOH, high NaOH, low temperature, high temperature, no incubation, long incubation, low NH₄OH, high NH₄OH, low CA & sorbitol, high CA & sorbitol. Asterisks (*) in the figure represent statistical differences compared to treatment number 1 with the level p<0.05 (Dunnett T3).

FIG. 8A illustrates sensory profiling of plant cell patties for odour. Error bars are standard errors of each attribute. From left to right: total odour intensity, sour berry intensity, biscuit odour intensity, other odour intensity. Treatments from left to right: AB-1 control, AB-5 high temperature, AB-2 low NaOH, AB-3 high NaOH, AB-10 no CA & sorbitol, R-3 high NaOH. Letters a, b, c and d represent statistical differences with the confidence level p<0.05 between samples in the two-way mixed model ANOVA.

FIG. 8B illustrates sensory profiling of plant cell patties for flavour. Error bars are standard errors of each attribute. From left to right: total flavor intensity, sourness, soapy or basic flavor, other flavor intensity. Treatments from left to right: AB-1 control, AB-5 high temperature, AB-2 low NaOH, AB-3 high NaOH, AB-10 no CA & sorbitol, R-3 high NaOH. Letters a and b represent statistical differences with the confidence level p<0.05 between samples in the two-way mixed model ANOVA.

FIG. 8C illustrates sensory profiling of plant cell patties for texture and mouthfeel. Error bars are standard errors of each attribute. From left to right: hardness, elasticity, loss of structure, solubility, biting resistance. Treatments from left to right: AB-1 control, AB-5 high temperature, AB-2 low NaOH, AB-3 high NaOH, AB-10 no CA & sorbitol, R-3 high NaOH. Letters a, b and c represent statistical differences with the confidence level p<0.05 between samples in the two-way mixed model ANOVA.

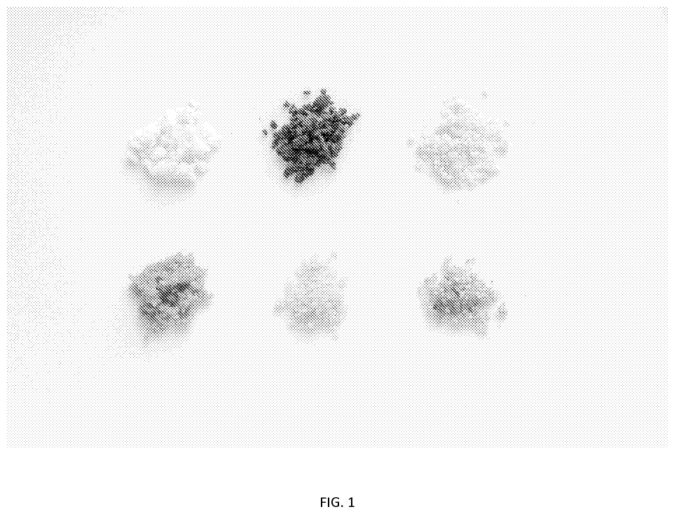

### DETAILED DESCRIPTION OF THE DISCLOSURE

This disclosure describes a process for converting biomass derived from a plant cell suspension culture into a three-dimensional plant biomass structure, obtaining a three-dimensional plant biomass structure.

The term “biomass” commonly refers to any substance of biotic origin. Biomass may be expressed as fresh weight, or as dry weight (excluding water contained in organisms).

The term “plant cell biomass” refers to plant cell broth i.e., plant cells or plant cell aggregates which have been separated from the nutrient medium in which they had been cultivated.

The term “plant biomass structure” refers to a structured plant cell biomass. Non-structured material i.e., brothy plant cell biomass material, which is not structured in any way, is harvested, and made to a structured material, “three-dimensional plant biomass structure”. Structured material may for example be in a form of foam-like “patty” or “patties”. The three-dimensional plant biomass structure may also be referred to as “a patty”, or “plant cell patty”, or “a solid foam patty”.

The plant cell broth is processed and formulated into three-dimensional shapes (3D shapes), i.e., “three-dimensional plant biomass structure” while preserving nutritional and sensory attributes without adding harmful (non-food grade) reagents. Plant cells are grown in suspension culture in a bioreactor. Non-structured material i.e., brothy cell biomass material, the plant cell mass i.e., the mixture comprising plant cells, which is not structured in any way, is harvested, and made to a structured material, such as foam-like patties. The patties may for example have a diameter in the range of 3-10 cm, for example 3, 4, 5, 6, 7, 8, 9, or 10 cm, or a range defined by any two of these values. The plant cell mass may be formulated into a three-dimensional shape. The “three-dimensional plant biomass structure” of the present disclosure is a stable three-dimensional (3D) plant biomass structure. In other words, the three-dimensional structure may pre-defined, self-supporting, durable and/or fixed three-dimensional structure.

“Plant cell mass” refers to plant cells or a plurality of plant cells, which are separated from the culture medium.

The term “porosity” or “porosity value” refers to the void (i.e., empty) spaces in a material and is a fraction of the volume of voids over the total volume, between 0 and 1, or as a percentage between 0% and 100%. There are many ways to test porosity in a substance or part, such as calculating from XRT images. Porosity is also referred to as void fraction. In the present disclosure an optional porosity value depends on the application. With a high porosity value, the patty, or a three-dimensional plant biomass structure is soft. With a low porosity value, the patty, or a three-dimensional plant biomass structure becomes hard and eventually film. In an embodiment of the present disclosure, the porosity value varies between 85% and 95%.

The term “hardness” or “hardness level” refers to the materials resistance to deformation.

The term “toughness” or “toughness level” refers to the total area under the force-deformation curve, measured in compression test analysis, describing the energy response of the material against the compression.

The present disclosure relates to the process for converting biomass derived from a plant cell suspension culture into a three-dimensional plant biomass structure, wherein the process comprises the steps of: providing a plant cell suspension culture comprising plant cells and culture medium; separating the plant cells from the culture medium to obtain plant cell mass; treating the plant cell mass with citric acid; optionally adding a humidifying agent to the plant cell mass; optionally adding water to the plant cell mass; optionally treating the plant cell mass with a base and adjusting pH of the plant cell mass to a desired level; incubating the plant cell mass at a temperature of 15 to 250° C. for 30 minutes to 24 hours; and dehydrating the plant cell mass to obtain a three-dimensional plant biomass structure.

The present disclosure relates to a process for converting biomass derived from a plant cell suspension culture into a three-dimensional plant biomass structure comprising the steps of providing a plant cell suspension culture comprising plant cells and culture medium; growing the plant cells in the culture medium; separating the plant cells from the culture medium by removing the culture medium to obtain plant cell mass; treating the plant cell mass with citric acid by mixing the plant cell mass and citric acid; formulating the plant cell mass into a three-dimensional shape; incubating the plant cell mass at a temperature of 15 to 250° C. for 30 minutes to 24 hours; and dehydrating the plant cell mass to obtain a three-dimensional plant biomass structure.

The present process for converting biomass derived from a plant cell suspension culture into a three-dimensional plant biomass structure comprises the following step: providing a plant cell suspension culture comprising plant cells and culture medium. The present process comprises growing the plant cells in the culture medium.

In “a plant cell culture (PCC)” or “a plant cell suspension culture” cells originate from a plant. Plant cells or plant material may originate from any plant or any plant cell culture. Also, mixtures of different plant cells can be used. Plant material or plant cells may originate for example from annual and/or perennial herbs, fruit-bearing plants, trees, and vegetables. Examples of suitable plants are rowan, lingonberry, arctic bramble, strawberry, cloudberry, raspberry, birch, avocado, chicory, sea buckthorn, and bilberry. Rowans, or mountain-ashes, belong to genus Sorbus of the rose family Rosaceae. Arctic bramble (Rubus arcticus), or arctic raspberry, belongs to genus Rubus of Rosaceae family.

A cell suspension or suspension culture is a type of cell culture in which single cells or small aggregates are allowed to function and multiply in an agitated culture or growth medium, thus forming a suspension. The cells can be derived from homogenized tissue or from non-homogenized tissue or from another type of culture. Depending on the host plant species, the size of cells and cell aggregates can vary between 50 μm to 2 mm. The size of the cells and aggregates may be in the range of 100 μm-1.5 mm, 200 μm-1 mm, or 500 μm-800 μm.

Particle size distribution of the plant cell suspension may be determined with any conventionally used method or equipment suitable for such determination, such as laser diffraction. Water may be used as a sample carrier; measurement range may be for example from 0.005-5000 μm. Fraunhofer approximation may be used to calculate the particle size distributions.

The plant suspension cell culture may be for example rowan suspension cell culture, arctic bramble suspension cell culture, strawberry suspension cell culture, lingonberry suspension cell culture, raspberry suspension cell culture, birch suspension cell culture, sea buckthorn suspension cell culture, or bilberry suspension cell culture.

Any conventionally used culture medium like MS medium (Murashige and Skoog 1962), or modified MS medium, or growth medium, which is suitable for cell suspension culture, can be used in the process. Preferably, the culture medium is liquid. Different types of media can be used for growing different types of cells. The media may be adjusted depending on the cell type. The growing conditions of the suspension cell culture may vary depending on the plant species.

The plant cell suspension cell culture may be grown in a suitable vessel. In an embodiment the plant suspension cell culture is grown in a bioreactor, which can be any suitable reactor or bioreactor. Examples of a bioreactor are a steel bioreactor, wave bioreactor or glass bioreactor, such as single-wall glass bioreactor. Growing callus on petri dish is not suitable for the present method.

The process also comprises the following step: separating the plant cells from the culture medium to obtain plant cell mass.

In an embodiment the plant cells are separated from the culture medium by removing the culture medium. The plant cells may be separated from the culture medium to obtain plant cell mass by filtration, centrifugation, sedimentation (gravity settling), or precipitation or a combination thereof. Plant cell mass contains plant cells or a plurality of plant cells. The culture medium is removed from cells e.g., by filtering for example by Buchner filtration.

In an embodiment the separated, e.g., filtered, plant cells, are weighted to match the dry content/total volume ratio between 1-10% dry content/total volume ratio, for example 2-9% dry content/total volume ratio, 3-8% dry content/total volume ratio, 4-7% dry content/total volume ratio, or 5-6% dry content/total volume ratio such as 1%, 2%, 3%, 4%, 5%, 6%, 7%, 8%, 9%, or 10%, or a ratio in the range defined by any two of these values. In a preferred embodiment filtered plant cells are weighted to match the 5.5% dry content/total volume ratio.

The process also comprises the following step: treating the plant cell mass with citric acid. In an embodiment the step comprises treating the plant cell mass with citric acid by mixing the plant cell mass and citric acid. In a preferred embodiment the plant cell mass and citric acid are mixed well.

Citric acid is an essential ingredient in the present process. Citric acid is added as a cross-linker. It facilitates crosslinking of cellulose molecules. Preferably, citric acid is a food grade ingredient. Citric acid may be added in an amount of 0-80 weight percent (wt %) of biomass dry weight content. The amount of citric acid may for example be in the range of 1-10, 1-20, 0-40, 1 40, 5-20, 5-40, 10-20, 10-40, 10-70, 20-40, 20-60, 1-80, 20-80, 30-50, or 30-80 weight percent (wt %) of biomass dry weight content, such as 1, 5, 10, 15, 20, 25, 30, 35, 40, 45, 50, 55, 60, 65, 70, 75, or 80 weight percent (wt %) of biomass dry weight content, or in the range defined by any two of these values.

The process may also comprise the following optional step: adding a humidifying agent to the plant cell mass.

A humidifier or humidifying agent or plasticizer may be added to the plant cell mass. Any food grade humidifier can be used. Humidifier is used for preventing a formation of brittle end products, i.e., patties or three-dimensional biomass structures. Examples of a humidifier are sorbitol, inositol, and glycerol. The humidifier is used to keep patties moist. The amount of humidifier may be 1-80 weight percent of biomass dry weight content. The amount of humidifier may for example be in the range of 1-3, 1-6, 1-13, 1-20, 3-6, 3-13, 3-20, 5-15, 5-20, 6-13, 10-70, 20-60, 20-80, 30-50, or 30-80 weight percent (wt %) of biomass dry weight content, such as 1, 3, 5, 6, 10, 13, 15, 20, 25, 30, 35, 40, 45, 50, 55, 60, 65, 70, 75, or 80 weight percent (wt %) of biomass dry weight content, or in the range defined by any two of these values.

In an embodiment plasticizer, e.g., sorbitol, is added in an amount of 0-13.333 wt % of samples dry content. The amount of plasticizer may be in a 1/3 (1:3) ratio to citric acid amount. The amount of plasticizer may vary between 1:0-1:1.

The plant cell mass, i.e., the mixture comprising plant cells, may be poured into a mold, shape or form. An example is a silicon cup. A skilled person in the art can select a suitable mold, shape or form.

The process may also comprise the following optional step: adding water to the plant cell mass. Water is added to facilitate the mixing or to reach the target volume.

The process may also comprise the following optional step: treating the plant cell mass with a base and adjusting pH of the plant cell mass to a desired level. Thus, the plant cell mass may be treated with a base to adjust pH of the plant cell mass to a desired level. The desired pH level may be between pH 2-pH 10. The pH of the plant cell mass may be between pH 3-9, pH 4-8, pH 5-7, such as pH 2, 3, 4, 5, 6, 7, 9, or 10, or in the range defined by any two of these values.

In an embodiment the plant cell mass is treated with a base. Any base, which is of food grade may be used. Different bases have different strengths; thus amount of base and type of base may vary.

The base may be selected from the group consisting of sodium hydroxide, sodium bicarbonate, ammonium bicarbonate, ammonia, potassium bicarbonate, calcium hydroxide, calcium carbonate, trisodium phosphate, sodium benzoate, and ammonium hydroxide and ammonium hydroxide (NH₄OH). Preferably, the base is selected from sodium bicarbonate (baking soda, NaHCO₃) and sodium hydroxide (lye, NaOH).

The choice of the base has an effect on the colour of the end product, i.e., the three-dimensional plant cell structure. The basic colour of the three-dimensional plant cell structure, however, originates from the suspension cell culture.

The process also comprises the following step: formulating the plant cell mass into a three-dimensional shape.

The plant cell mass may be place into a mold, shape or form. For example, the plant cell mass may be poured into a mold. An example of a mold is a silicon cup. A skilled person in the art can select a suitable mold, shape or form. The shape of the three-dimensional structure depends on the shape of the mold or form. The “three-dimensional plant biomass structure” of the present disclosure is a stable three-dimensional (3D) plant biomass structure. In other words, the three-dimensional structure may pre-defined, self-supporting, durable and/or fixed three-dimensional structure.

The process also comprises the following step: incubating the plant cell mass at a temperature from 15 to 250° C. for 0 to 24 hours, preferably for 30 minutes (i.e., 0.5 hours) to 24 hours. The incubation temperature may for example be in the range of 15-250° C., 20-80° C., 20-100° C., 20-140° C., 20-240° C., 30-230° C., 40-220° C., 50-210° C., 60-200° C., 70-190° C., 80-140° C., 80-180° C., 90-170° C., 100-160° C., 110-150° C., or 120-140° C., such as 15, 20, 30, 40, 50, 60, 70, 80, 90, 100, 110, 120, 130, 140, 150, 160, 170, 180, 190, 200, 210, 220, 230, 240, or 250° C., or in the range defined by any two of these values. The incubation time may for example be 0-24 hours, 30 minutes-24 hours, 30 minutes-1 hour, 30 minutes-2 hours, 30 minutes-3 hours, 30 minutes-4 hours, 1-4 hours, 1-10 hours, 1-24 hours, 2-24 hours, 3-24 hours, 4-23 hours, 5-21 hours, 6-20 hours, 7-19 hours, 8-18 hours, 9-16 hours, 10-15 hours, 11-14 hours, 12-13 hours, or 30 minutes, 1, 2, 3, 4, 5, 6, 7, 8, 9, 10, 11, 12, 13, 14, 15, 16, 17, 18, 19, 20, 21, 22, 23, or 24 hours, or in the range defined by any two of these values. Incubation temperature options listed above can be combined with incubation time options above. An optional incubation time depends on the application. When incubating at higher temperature shorter incubation time is used to avoid burning of the sample. When incubating at lower temperature longer incubation time can be used. The temperature may be a room temperature (RT).

In a preferred embodiment, the plant cell mass is mixed well before the incubation. In a preferred embodiment, the incubation time is at least 30 minutes.

In an embodiment the plant cell mass is incubated at 15-140° C. or at room temperature −140° C., for 30 minutes-4 hours.

Incubation conditions define the final form of the three-dimensional plant cell structure. The final form may for example be in a form of sponge or film. For sponge like structure, either shorter incubation times or lower incubation temperatures are used to prevent the too high evaporation of the water before freezing and freeze drying. For film like structure, samples kept longer in incubation or higher incubation temperatures are used to evaporate almost all the water from the sample. For film like structures freezing and freeze drying are not necessary.

The process also comprises the following step: dehydrating the plant cell mass.

The plant cell mass is dried or dehydrated. A skilled person can choose a suitable dehydration method for use in the present method. For example, dehydration methods for preservation of food by drying in the food processing industry can be used. Drying methods can be classified based on the water-removing method applied, such as thermal drying, osmotic dehydration, and mechanical dewatering. Thermal drying includes such as air drying, vacuum drying, low air environment drying and modified atmosphere drying.

For example, freeze drying or oven drying may be used for dehydration of the plant cell mass. Preferably, the plant cell mass is dehydrated by freeze drying. Freeze drying is also known as lyophilization or cryodesiccation or low temperature dehydration process. The length of dehydration time depends on the size of the machine or equipment used for the dehydration and the amount of water to be removed.

In an embodiment the process may also comprise the following step if a mold, shape or form is used to formulate the plant cell mass into a three-dimensional shape: removing the dehydrated plant cell mass from the mold, shape or form. The process may also comprise the following step: obtaining a three-dimensional plant biomass structure.

Thus, a three-dimensional plant biomass structure is obtained as a result of the present process. The obtained three-dimensional plant biomass structure may be of any shape. The shape of the three-dimensional structure depends on the shape of the mold or form. The shape of the three-dimensional plant biomass structure may for example be cylinder, cube, sphere, hemisphere, octahedron, or cone. The shape may alternatively be triangular pyramid, rectangular pyramid, hexagonal pyramid, or pentagonal pyramid. As a further alternative, the shape may for example be hexagonal prism, triangular prism, pentagonal prism of quadrangular prism. The shape of the three-dimensional plant biomass structure may be an irregular shape.

The three-dimensional structures of the present disclosure are obtained by cross-linking the cell wall structures, such as cellulose and pectin, with citric acid through the disclosed process. The present process involves chemical crosslinking and hydrolysis of plant cell wall components, such as cellulose in the primary cell wall and pectin in the middle lamella. The polysaccharide groups are crosslinked with citric acid, including partial alkaline hydrolysis, followed by dehydration, such as freeze drying. Individual plant cells and cell aggregates form structured foam patties, i.e., three-dimensional plant biomass structures.

The disclosure also describes a three-dimensional plant biomass structure obtained by presently described process, wherein the structure has a porosity value between 85-95% and at least one property selected from the group consisting of: toughness level between 13-185 kg*mm and hardness level between 6-108 kg.

Porosity values of the three-dimensional plant biomass structure may for example be in the range of 85-95%, such as 85%, 86%, 87%, 88%, 89%, 90%, 91%, 92%, 93%, 94% or 95%, or a range defined by any two of these values. Porosity values can be measured with any conventionally used method or device which is suitable for such measurement. For example, porosity values of the three-dimensional plant biomass structure can be calculated from XRT images. In the present disclosure an optional porosity value depends on the application. With a high porosity value, the patty, or a three-dimensional plant biomass structure is soft. With a low porosity value, the patty, or a three-dimensional plant biomass structure becomes hard and eventually film

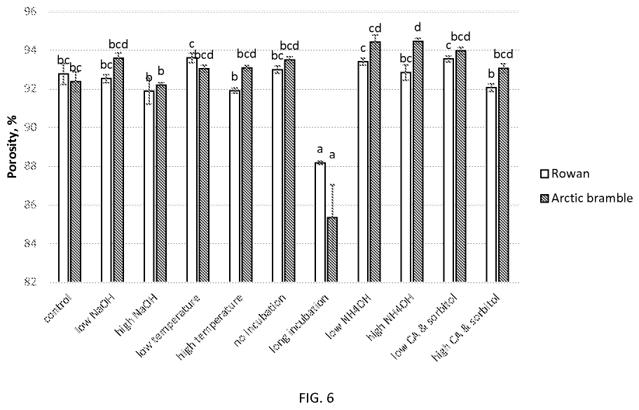

In an embodiment of the present disclosure porosity values calculated from XRT images vary between 88-95% for rowan patties or three-dimensional rowan biomass structures (FIG. 6).

In another embodiment of the present disclosure porosity values calculated from XRT images vary 85-95% for arctic bramble patties or three-dimensional arctic bramble biomass structures (FIG. 6).

Toughness levels may for example be in the range of 13-185 kg*mm, 20-185 kg*mm, 30-185 kg*mm, 40-185 kg*mm, 50-185 kg*mm, 60-185 kg*mm, 70-185 kg*mm, 80-185 kg*mm, 90-185 kg*mm, 100-185 kg*mm, 110-185 kg*mm, 120-185 kg*mm, 130-185 kg*mm, 140-185 kg*mm, 150-185 kg*mm, 160-185 kg*mm, 170-185 kg*mm or 180-185 kg*mm. Toughness levels can be measured with any conventionally used method of device which is suitable for such measurement. For example, toughness levels can be extracted from a force-deformation curve. Toughness is calculated from the area under the force-deformation curve, as the energy response of the material against the compression (Sozer et al., 2011).

Hardness levels of the three-dimensional plant biomass structure may be in the range of 6-108 kg, 10-108 kg, 20-108 kg, 30-108 kg, 40-108 kg, 50-108 kg, 60-108 kg, 70-108 kg, 80-108 kg, 90-108 kg, or 100-108 kg.

Any porosity value option listed above can be combined with any toughness level option above. Any toughness level option listed above can be combined with any hardness time option above. Any porosity value option listed above can be combined with any hardness option above.

The above parameters may be interlinked. Parameters depend on the purpose where the product is used. The qualities and quantities of the three-dimensional plant biomass structure may be adjusted according to the end product.

The process of the present disclosure enables downstream processing and formulation of plant cell biomass (i.e., cell broth) into three-dimensional shapes while preserving nutritional and sensory attributes without adding potentially harmful (non-food grade) reagents. Plant cells are grown in suspension culture in a bioreactor. Non-structured material i.e., brothy cell biomass material, which is not structured in any way, is harvested, and made to a structured material, such as foam-like patties, for example having a diameter of ca. 5 cm.

The disclosure also describes a food product comprising the three-dimensional plant biomass structure, wherein the food product comprises one or more other or further ingredients selected from the group consisting of proteins, fats, polysaccharides, colourings, flavourings, preservatives, salt, antioxidants, and sugars.

The plant biomass structure of the present disclosure may replace an ingredient or ingredients in the food product.

The food product may for example be a bar, snack, snack bar, muesli, cracker, chip, topping, garnish, or decoration. The three-dimensional plant biomass structure may be directly used as a food product, for example as a bar.

The disclosure also describes the use of a three-dimensional plant biomass structure as an ingredient in a food product. The three-dimensional plant biomass structure may be used as a sole ingredient in the food product, or in addition to another ingredient or ingredients in the food product.

The three-dimensional plant biomass structure may be added to or mixed with a conventional product. The three-dimensional plant biomass structure may be amended or mixed with another ingredient such as a protein. The three-dimensional plant biomass structure may be added to an ingredient e.g., to muesli.

In an embodiment the process of the present disclosure relates to a process for converting biomass derived from a plant cell suspension culture into a three-dimensional plant biomass structure, wherein the process comprises the steps of: providing a plant cell suspension culture comprising plant cells and culture medium; separating the plant cells from the culture medium to obtain plant cell mass; treating the plant cell mass with citric acid; optionally adding a humidifying agent to the plant cell mass; optionally adding water to the plant cell mass; optionally treating the plant cell mass with a base and adjusting pH of the plant cell mass to a desired level; incubating the plant cell mass at a temperature of 15 to 250° C. for 0 to 24 hours; dehydrating the plant cell mass; and obtaining a three-dimensional plant biomass structure.

In an embodiment the process of the present disclosure relates to a process for converting biomass derived from a plant cell suspension culture into a three-dimensional plant biomass structure, wherein the process comprises the steps of: providing a plant cell suspension culture comprising plant cells and culture medium; separating the plant cells from the culture medium to obtain plant cell mass; treating the plant cell mass with citric acid; optionally adding a humidifying agent to the plant cell mass; optionally adding water to the plant cell mass; optionally treating the plant cell mass with a base and adjusting pH of the plant cell mass to a desired level; incubating the plant cell mass at a temperature of 15 to 250° C. for 30 minutes to 24 hours; and dehydrating the plant cell mass to obtain a three-dimensional plant biomass structure.

In an embodiment the process of the present disclosure relates to a process for converting biomass derived from a plant cell suspension culture into a three-dimensional plant biomass structure, wherein the process comprises the steps of: providing a plant cell suspension culture comprising plant cells and culture medium; separating the plant cells from the culture medium to obtain plant cell mass; treating the plant cell mass with citric acid; adding a humidifying agent to the plant cell mass; optionally adding water to the plant cell mass; optionally treating the plant cell mass with a base and adjusting pH of the plant cell mass to a desired level; incubating the plant cell mass at a temperature of 15 to 250° C. for 30 minutes to 24 hours; and dehydrating the plant cell mass; to obtain a three-dimensional plant biomass structure.

In an embodiment the process of the present disclosure relates to a process for converting biomass derived from a plant cell suspension culture into a three-dimensional plant biomass structure, wherein the process comprises the steps of: providing a plant cell suspension culture comprising plant cells and culture medium; separating the plant cells from the culture medium to obtain plant cell mass; treating the plant cell mass with citric acid; optionally adding a humidifying agent to the plant cell mass; optionally adding water to the plant cell mass; treating the plant cell mass with a base and adjusting pH of the plant cell mass to a desired level; incubating the plant cell mass at a temperature of 15 to 250° C. for 30 minutes to 24 hours; and dehydrating the plant cell mass; to obtain a three-dimensional plant biomass structure.

In an embodiment the process of the present disclosure relates to a process for converting biomass derived from a plant cell suspension culture into a three-dimensional plant biomass structure, wherein the process comprises the steps of: providing a plant cell suspension culture comprising plant cells and culture medium; separating the plant cells from the culture medium to obtain plant cell mass; treating the plant cell mass with citric acid; adding a humidifying agent to the plant cell mass; optionally adding water to the plant cell mass; treating the plant cell mass with a base and adjusting pH of the plant cell mass to a desired level; incubating the plant cell mass at a temperature of 15 to 250° C. for 30 minutes to 24 hours; and dehydrating the plant cell mass to obtain a three-dimensional plant biomass structure.

In an embodiment the process of the present disclosure relates to a process for converting biomass derived from a plant cell suspension culture into a three-dimensional plant biomass structure, characterized in that the process comprises the steps of: providing a plant cell suspension culture comprising plant cells and culture medium; separating the plant cells from the culture medium to obtain plant cell mass; treating the plant cell mass with citric acid; incubating the plant cell mass at a temperature of 15 to 250° C. for 30 minutes to 24 hours; dehydrating the plant cell mass; and obtaining a three-dimensional plant biomass structure.

In an embodiment the process of the present disclosure relates to a process for converting biomass derived from a plant cell suspension culture into a three-dimensional plant biomass structure, characterized in that the process comprises the steps of: providing a plant cell suspension culture comprising plant cells and culture medium; separating the plant cells from the culture medium to obtain plant cell mass; treating the plant cell mass with citric acid; adding a humidifying agent to the plant cell mass; adding water to the plant cell mass; treating the plant cell mass with a base and adjusting pH of the plant cell mass to a desired level; incubating the plant cell mass at a temperature of 15 to 250° C. for 30 minutes to 24 hours; dehydrating the plant cell mass; and obtaining a three-dimensional plant biomass structure.

In an embodiment the process of the present disclosure relates to a process for converting biomass derived from a plant cell suspension culture into a three-dimensional plant biomass structure, wherein the process comprises the steps of: providing a plant cell suspension culture comprising plant cells and culture medium; growing the plant cells in the culture medium; separating the plant cells from the culture medium by removing the culture medium to obtain plant cell mass; treating the plant cell mass with citric acid by mixing the plant cell mass and citric acid; optionally adding a humidifying agent to the plant cell mass; optionally adding water to the plant cell mass; optionally treating the plant cell mass with a base and adjusting pH of the plant cell mass to a desired level; formulating the plant cell mass into a three-dimensional shape; incubating the plant cell mass at a temperature of 15 to 250° C. for 30 minutes to 24 hours; dehydrating the plant cell mass to obtain a three-dimensional plant biomass structure.

In an embodiment the process of the present disclosure relates to a process for converting biomass derived from a plant cell suspension culture into a three-dimensional plant biomass structure, wherein the process comprises the steps of: providing a plant cell suspension culture comprising plant cells and culture medium; growing the plant cells in the culture medium; separating the plant cells from the culture medium by removing the culture medium to obtain plant cell mass; treating the plant cell mass with citric acid by mixing the plant cell mass and citric acid; adding a humidifying agent to the plant cell mass; adding water to the plant cell mass; treating the plant cell mass with a base and adjusting pH of the plant cell mass to a desired level; formulating the plant cell mass into a three-dimensional shape; incubating the plant cell mass at a temperature of 15 to 250° C. for 30 minutes to 24 hours; dehydrating the plant cell mass to obtain a three-dimensional plant biomass structure.

In an embodiment the process of the present disclosure relates to a process for converting biomass derived from a plant cell suspension culture into a three-dimensional plant biomass structure, wherein the process comprises the steps of: providing a plant cell suspension culture comprising plant cells and culture medium; growing the plant cells in the culture medium; separating the plant cells from the culture medium by removing the culture medium to obtain plant cell mass; treating the plant cell mass with citric acid by mixing the plant cell mass and citric acid; optionally adding a humidifying agent to the plant cell mass; optionally adding water to the plant cell mass; optionally treating the plant cell mass with a base and adjusting pH of the plant cell mass to a desired level; placing the plant cell mass into a mold; incubating the plant cell mass in the mold at a temperature of 15 to 250° C. for 30 minutes to 24 hours; dehydrating the plant cell mass; and removing the dehydrated plant cell mass from the mold to obtain a three-dimensional plant biomass structure.

In an embodiment the process of the present disclosure relates to a process for converting biomass derived from a plant cell suspension culture into a three-dimensional plant biomass structure, wherein the process comprises the steps of: providing a plant cell suspension culture comprising plant cells and culture medium; growing the plant cells in the culture medium; separating the plant cells from the culture medium by removing the culture medium to obtain plant cell mass; treating the plant cell mass with citric acid by mixing the plant cell mass and citric acid; placing the plant cell mass into a mold; incubating the plant cell mass in the mold at a temperature of 15 to 250° C. for 30 minutes to 24 hours; dehydrating the plant cell mass; and removing the dehydrated plant cell mass from the mold to obtain a three-dimensional plant biomass structure.

In an embodiment the process of the present disclosure relates to a process for converting biomass derived from a plant cell suspension culture into a three-dimensional plant biomass structure, which a plant cell patty, wherein the process comprises the steps of providing a plant cell suspension culture comprising plant cells and culture medium; growing the plant cells in the culture medium; separating the plant cells from the culture medium by removing the culture medium to obtain plant cell mass; treating the plant cell mass with citric acid by mixing the plant cell mass and citric acid; formulating the plant cell mass into a three-dimensional shape; incubating the three-dimensional plant cell mass in a shape at a temperature of 15 to 250° C. for 30 minutes to 24 hours; and dehydrating the plant cell mass in the shape to obtain a plant cell patty.

In an embodiment of the present process the plant cells are separated from the culture medium by filtration, centrifugation, sedimentation, or precipitation or a combination thereof.

In an embodiment of the present process the base is selected from the group consisting of sodium hydroxide, sodium bicarbonate, ammonium bicarbonate, ammonia, potassium bicarbonate, calcium hydroxide, calcium carbonate, trisodium phosphate, sodium benzoate, and ammonium hydroxide.

In an embodiment the plant cell mass is dehydrated by freeze drying.

An aspect of the present disclosure is a three-dimensional plant biomass structure obtained by the present process and having at least one property selected from the group consisting of: a porosity value between 85-95%, toughness level between 13-185 kg*mm, and hardness level between 6-108 kg.

In an embodiment a three-dimensional plant biomass structure obtained by the present process has a porosity value between 85-95%, and at least one property selected from the group consisting of: toughness level between 13-185 kg*mm and hardness level between 6-108 kg.

In an embodiment a three-dimensional plant biomass structure obtained by the present process has a porosity value between 85-95%, toughness level between 13-185 kg*mm and hardness level between 6-108 kg.

In an embodiment of the disclosure the three-dimensional plant biomass structure is a plant cell patty.

An aspect of the present disclosure is a food product comprising the three-dimensional plant biomass structure wherein the food product comprises one or more further ingredients selected from the group consisting of proteins, fats, polysaccharides, colourings, flavourings, preservatives, salt, antioxidants, and sugars.

In an embodiment of the present disclosure the food product is selected from the group consisting of a bar, snack, snack bar, muesli, cracker, chip, topping, garnish, and decoration.

An aspect of the present disclosure is a use of the three-dimensional plant biomass structure as an ingredient in a food product. In an embodiment the three-dimensional plant biomass structure is used as a sole ingredient. In an embodiment the three-dimensional plant biomass structure is used in addition to or with another ingredient.

In an embodiment the present disclosure relates to a method for structuring biomass derived from plant cell suspension culture, wherein the method comprises generating plant material comprising plant suspension cell culture, separating cell mass from plant cell suspension culture medium, removing the medium from cells, preferably by filtering, treating the plant cell mass with citric acid, dehydrating the cells/cell material, preferably by freeze drying.

The present disclosure relates to a method of chemically crosslinking biomass derived from plant cell cultures for food applications. The present disclosure provides a method of converting biomass derived from a plant cell culture into three-dimensional form while preserving nutritional value, colour and flavor. The present disclosure provides a method of structuring plant cell culture-derived biomass broth in a simple and food grade way and preserving nutritional value, colour and flavour.

In the present process wet plant cell biomass is converted into three-dimensional form, such as dry film, or spongy structure. Features of the plant cell biomass can be modulated by treatment conditions, and this opens various applications for example in food industry.

In the present disclosure a novel way of food architecture design by chemical cross-linking and freeze-drying of plant cells is presented. Cellulose and pectin, forming the primary cell wall and middle lamella, respectively, are exploited as a structural basis. Carboxyl groups of polysaccharide chains are cross-linked by using citric acid, followed by freeze drying where individual plant cells and cell aggregates form structured solid foam patties made of plant cells. Change in structural and textural properties are followed by uni-axial compression test, microscopy imaging and x-ray micro-computed tomography (XμCT) as a function of additives and incubation conditions.

The mechanical properties of PCPs (plant cell patties) are measured after the sample relative humidity % (rh %) reaches equilibrium by compression test in TA XT Plus Texture Analyser equipped with a 30 kg load cell, or by TA-HDi Texture Analyser with 250 kg load cell. Textural parameters may be evaluated based on the resulted force-deformation curves.

Additionally, a trained sensory panel evaluates the taste and mouth feel of the samples. To ensure the safety of samples tasted in sensory evaluations, microbiological analyses are carried out. Samples are taken from the cultivation suspension and from plant cell patties after freeze drying. The sensory properties of the samples are evaluated with generic descriptive analysis. The odor attributes are for example total odor intensity, sour berry odor intensity, biscuit odor intensity and other odor intensity. The tactile texture attributes are for example hardness and elasticity, both evaluated by using fingers. Taste and flavor attributes are total flavor intensity, sourness, soapy/basic flavor, and other flavor intensity. Mouthfeel attributes consist of loss of structure, solubility and biting resistance.

In an embodiment plant cells low in starch [0.3-1.3% dry weight (dw)] but rich in both dietary fibre (21-37% dw) and protein (12-23% dw) can provide new opportunities for the food ingredient and manufacturing industries. Plant cell cultures are source of good quality lipids (e.g., linoleic, and α-linoleic acid) with a well-balanced amino acid profile, including all essential amino acids.

It is apparent to a person skilled in the art that as technology advances, the basic idea of the invention can be implemented in various ways. The invention and its embodiments are therefore not restricted to the above examples, but they may vary within the scope of the claims.

As shown in the examples, the present inventors were able to successfully produce three-dimensional plant biomass structures. The obtained plant biomass structures can be used in food products as a sole ingredient or as an additional ingredient.

### Examples

### Methods

### Plant Cell Lines and Culture Protocols

Plant cell suspension cultures (PCCs) of rowan (Sorbus aucuparia L.; VTTCC P-12000) and arctic bramble (Rubus arcticus; VTTCC P-120088) were used for plant cell patty experiments. The cell cultures were maintained and cultivated as previously described (Suvanto et al. 2017). Briefly, cell suspension was grown in 250 ml Erlenmeyer flask containing 70 ml of culture on an orbital shaker at 110 rpm, 24° C.

Rowan was cultivated in Murashige & Skoog (MS) medium including vitamins (Duchefa Biochemie, Haarlem, The Netherlands) (pH 4.8) supplemented with 0.1 mg/L kinetin (Sigma Aldrich, St Louis, USA) and 1 mg/L α-naphthaleneacetic acid (Sigma Aldrich, St Louis, USA) in darkness. The subculturing rhythm was 7 days.

Arctic bramble was cultivated in modified MS medium including vitamins (pH 5.8) with the same hormone additions of kinetin (0.1 mg/L) (Sigma Aldrich, USA) and α-naphthaleneacetic acid (1 mg/L) (Sigma Aldrich, USA), applying a 10 d subculturing rhythm and day-night light regime (16:8 h photoperiod, irradiation 40 μmol m⁻² s⁻¹).

For biomass production, rowan cells were cultivated in a wave bioreactor (Biostat RM, Sartorius, Germany) using 20 L wave bags (Cultibag R M, 20 L basic wave bag, Sartorius Stedim, Gottingen, Germany) and the following cultivation parameters: temperature 24° C., rocking level 22, angle 10°, aeration 300 ml/min, in darkness. An inoculum of 30 g/L was prepared from shake flask or previous wave cultures. The growth time in the wave bioreactor was set to 10 days.

Arctic bramble biomass was cultivated in a 5 L single-wall glass bioreactor (BioFlo320, Eppendorf, Hamburg, Germany) and the following cultivation parameters: working volume 5 L, temperature 24° C., dissolved oxygen target 20-15%, aeration 0.8 lpm (liters per minute), stirring at 50-250 rpm (marine impeller, 3-blade). Continuous white light (20% RGB control) was provided by two LED-panels (SL 3500 LED-panels, Photon Systems Instruments, Drasov, Czech Republic) on opposite sides of the glass bioreactor. Inoculum of 30 g/L was prepared from shake-flask cultures. The growth time in the bioreactor was set to 14 days.

Plant cells were harvested by filtering with Miracloth (Calbiochem, San Diego, CA, USA) in a Buchner funnel and were stored at +8° C. until plant cell patty preparation. The dry weight/fresh weight ratio of each batch of filtered cells was determined for standardization.

### Particle Size Analysis

Particle size distribution of the plant cell suspension cultures was determined by laser diffraction using a Malvern Mastersizer 3000 instrument with Hydro LV liquid dispersion unit (Malvern Instruments, Worcestershire, UK). Water was used as a sample carrier; the measurement range was set to 0.005-5000 μm, and measurements were taken at a mixing speed of 2000 rpm. Fraunhofer approximation was used to calculate the particle size distributions. Samples were analysed as duplicates with five parallel measurements per run.

### Plant Cell Patty Preparation

The total volume of a plant cell patty was 45 mL before incubation. Filtered cells were weighted to achieve the 5.5% dry mass/total volume ratio in order to avoid batch-to-batch variations. 20% citric acid was added as cross-linker (up to 40 wt % of the dry cell mass). Sorbitol was added as plasticizer—(up to 13.3 wt %, but always one third of the amount of citric acid. For some samples 1 M NaOH or 1 M NH₄OH was added (Table 1). The total volume was made up by adding Milli-Q water. After mixing the samples, pH and conductivity were measured, and samples were incubated in silicon cups (diameter 6 cm) for 0-4 h at temperatures of 20° C. (low), 80° C. (normal) or 140 (high) ° C. Exact conditions for each treatment are presented in Table 1. Replicate patties for each treatment were prepared using cells from the same culture batch. Following incubation, the samples were freeze dried. Freeze dried samples were placed in a desiccator with at 43% relative humidity (saturated potassium carbonate solution) until the relative product humidity in the samples reached equilibrium as determined by water activity measurements using aCX-2 water activity meter, AQUAlab, H6 hr-Grenzhausen, Germany). Samples were prepared for sensory evaluation using food-grade or pharma-grade chemicals.

### Color Measurement

The color of cell biomass and patty material was measured using a CR-200 chromo-meter (Minolta, Osaka, Japan) calibrated with a CR-A43 white ceramic plate. For fresh and freeze-dried cell material, a small Petri dish was filled with the sample and color was measured in three replicate samples at five different points each. For patties, the upper side of a patty was placed against a Petri dish and the color was measured at five different points per patty. Color was measured after samples reached equilibrium (43% relative humidity). The color was recorded as coordinates in the CIE Lab color space. The total color difference (ΔE) between control patty and other treatments was defined using following equation:

$$Δ E = √ [ {( {L}_{c} - {L}_{s} )}^{2} ) + ( {a}_{c} - {a}_{s} ) 2 ) + {( {b}_{c} - {b}_{s} )}^{2} ) ]$$

where c refers to the control patty and s to other patty samples.

### Microbiological Assays

To ensure the safety of samples tasted in sensory evaluations, microbiological analyses were carried out. Samples were taken from cell suspension cultures during cultivation and from plant cell patties after freeze drying. From patties, 100 mg of freeze-dried material was diluted 10⁻² and 10⁻³ in 9 ml of sterile peptone saline solution, and 100 μl of the diluted solution was transferred to plate count agar (PCA) and potato dextrose agar (PDA) plates. The plates were incubated for 3 d at 28° C. and for 5 d at 25° C., respectively. For sensory evaluation, the microbial count threshold was set to 10 colony forming units (CFU)/g or 1 CFU/ml.

### Mechanical Properties

The mechanical properties of PCPs (plant cell patties) were measured after the sample reached equilibrium (43% relative humidity) A uniaxial compression test was carried out using in TA XT Plus Texture Analyser (Stable Micro Systems, Godalming, UK) equipped with a 30-kg load cell, except samples from treatment R-7 (rowan, long incubation time) which were measured using a TA-HDi Texture Analyser (Stable Micro Systems, UK) with 250-kg load cell. A 100 mm compression plate was used as the probe at a constant speed of 2 mm/s and 70% strain, with triggering force of 10 g. Data were collected at a rate of 200/second (pps) and were processed using Exponent stable micro system software version v6.1.16.0. Textural parameters were evaluated based on the force-deformation curves and results were reported as the average of the four replicates per condition. Hardness was recorded as a maximum force after 70% strain compression. Toughness is calculated from the area under the force-deformation curve, as the energy response of the material against the compression (Sozer et al., 2011.

### X-Ray Micro-Computed Tomography (XμCT)

For examining the 3D structure of plant cell patties, XμCT imaging method was used. Chosen plant cell patty samples were cut into 10×10×10 mm blocks and were imaged using Rx Solutions desktop 130 scanner at an acceleration voltage of 40 kV with a voxel size of 3.7 μm. Scanning time was 15 minutes per sample. Each sample was scanned in triplicate. The thicknesses of structures was measured using a local thickness transform, which can determine the pore size and distribution as well as the wall thickness (Hildebrand and Rüegsegger 1997) was used.

### Sensory Profiling

The sensory properties of the samples were evaluated with generic descriptive analysis. The sensory evaluation panel consisted of six assessors from the food and beverage trained sensory panel of VTT Technical Research Centre of Finland Ltd. The small panel size was a safety precaution due to COVID-19. The sensory evaluation was performed at VTT's sensory evaluation laboratory which fulfils the requirements of the ISO standards (ISO 6658, 2017 and ISO 8589:2007). All assessors on the sensory panel have been tested regarding gustation, olfaction and color vision and trained in sensory methods. The necessary individual information of the panel was collected in following the guidelines of the EU General Data Protection Regulation GDPR (2016/679). An application regarding the sensory evaluation was made to the ethical committee of VTT, who reviewed and recommended the evaluation protocol and safety guidelines. The assessors provided written informed consent before the evaluations.

Descriptive sensory profiling was done with 13 attributes, of which six had reference products with bound intensities. All attributes were well defined and described verbally and the ends of intensity scales were anchored verbally. Line scales from 0=no attribute to 10=very clear attribute were used to evaluate the intensities. The sensory attributes covered the odor, appearance, texture, taste, flavor, and mouthfeel characteristics of the samples.

The odor attributes were total odor intensity, sour berry odor intensity, biscuit odor intensity and other odor intensity. The tactile texture attributes were hardness and elasticity, both evaluated by using fingers. Taste and flavor attributes were total flavor intensity, sourness, soapy/basic flavor, and other flavor intensity. Mouthfeel attributes consisted of loss of structure, solubility and biting resistance. Evaluations were performed with duplicate sessions. The samples were coded with 3-digit codes and a complete block design with a Latin square randomized sample serving order were used.

The sample portion for each assessor consisted of three pieces, cut into 10×10×10 mm sample cubes, served in plastic sample vials with a lid. Following the safety guidelines of the ethical committee, the assessors were instructed not to swallow the samples but to spit them out after tasting.

### Statistical Analyses

Statistical analyses were conducted with IBM Statistics 26 software. Normality of data was evaluated with Shapiro-Wilk test. One-way analysis of variance (ANOVA) was used with Tukey's honest significant difference (HSD) or Dunnett T3 post-hoc test, depending on the data variance. For the sensory profiling data, a two-way mixed model ANOVA with samples as the fixed factor and assessors as a random factor, with Tukey's HSD post-hoc test was used. A confidence level p<0.05 was used for all statistical analysis.

### Results

### Characterization of Plant Cells

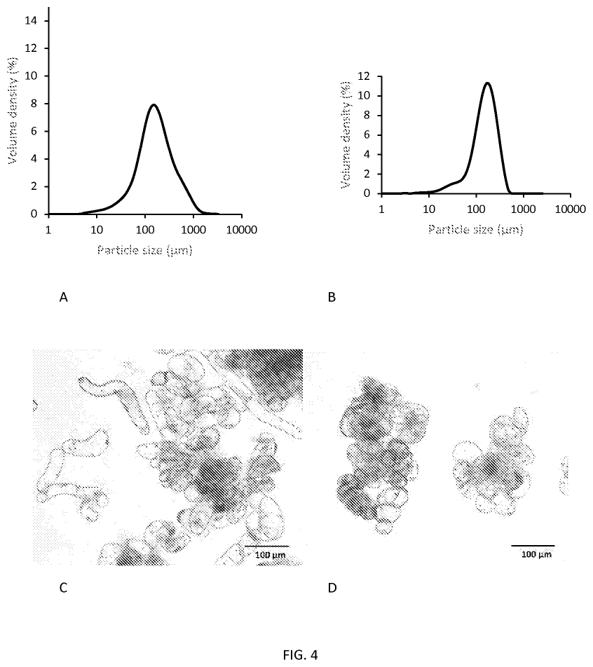

Cell suspensions of two plant cell lines rowan, and arctic bramble, were investigated as a raw material for the structuring experiments. Depending on the growth stage, rowan cultures contained suspended cells displaying round, oval, and elongated shapes (FIG. 4C). On the contrary, suspended arctic bramble cells were uniform and mainly appeared round-shaped (FIG. 4D). Both cell lines tended to form cell aggregates consisting of cells attached to each other. Although most aggregates in both cultures measured ca. 100 μm, the size distribution was cell line specific (FIGS. 4A, 4B). Filtered fresh biomass was a smooth broth containing 92% water.

### Structure of Plant Cell Patties

The plant cell biomass was structured by varying the pH, amount of citric acid and sorbitol, the base, the incubation time, and the temperature. Twelve different treatments (Table 1) to structure the cell culture-derived biomass were tested for each cell line. Filtered cells and cell aggregates were incubated for 0, 1 or 4 h at temperatures of 20, 80 or 140° C., followed by freeze drying, which converted the biomass into dry, soft, and porous structures termed plant cell patties.

TABLE 1
Conditions to structure cell culture-derived biomass. In each treatment, one parameter compared to
the control sample was modified to investigate the effects on the structure of the patty. (n = 4).

| Plant | Treatment | Treatment | Sample | Citric acid, | Sorbitol, | Added | Incubation | Incubation |
|---|---|---|---|---|---|---|---|---|
| cell line | explanation | number | code | wt % | wt % | base, mL | time, h | temperature, ° C. |
| Rowan | Control | 1 | R-1 | 20 | 6 | — | 1 | 80 |
| | NaOH addition | 2 | R-2 | 20 | 6 | NaOH, | 1 | 80 |
| | | | | | | 2.5 mL | | |
| | | 3 | R-3 | 20 | 6 | NaOH, | 1 | 80 |
| | | | | | | 7.5 mL | | |
| | Incubation | 4 | R-4 | 20 | 6 | — | 1 | 20 |
| | temperature | 5 | R-5 | 20 | 6 | — | 1 | 140 |
| | Incubation time | 6 | R-6 | 20 | 6 | — | 0 | 80 |
| | | 7 | R-7 | 20 | 6 | — | 4 | 80 |
| | NH₄OH addition | 8 | R-8 | 20 | 6 | NH₄OH, | 1 | 80 |
| | | | | | | 2.5 mL | | |
| | | 9 | R-9 | 20 | 6 | NH₄OH, | 1 | 80 |
| | | | | | | 7.5 mL | | |
| | Citric acid and | 10 | R-10 | 0 | 0 | — | 1 | 80 |
| | sorbitol wt % | 11 | R-11 | 10 | 3 | — | 1 | 80 |
| | | 12 | R-12 | 40 | 13 | — | 1 | 80 |
| Arctic | Control | 1 | AB-1 | 20 | 6 | — | 1 | 80 |
| Bramble | NaOH addition | 2 | AB-2 | 20 | 6 | NaOH, | 1 | 80 |
| | | | | | | 2.5 mL | | |
| | | 3 | AB-3 | 20 | 6 | NaOH, | 1 | 80 |
| | | | | | | 7.5 mL | | |
| | Incubation | 4 | AB-4 | 20 | 6 | — | 1 | 20 |
| | temperature | 5 | AB-5 | 20 | 6 | — | 1 | 140 |
| | Incubation time | 6 | AB-6 | 20 | 6 | — | 0 | 80 |
| | | 7 | AB-7 | 20 | 6 | — | 4 | 80 |
| | NH₄OH addition | 8 | AB-8 | 20 | 6 | NH₄OH, | 1 | 80 |
| | | | | | | 2.5 mL | | |
| | | 9 | AB-9 | 20 | 6 | NH₄OH, | 1 | 80 |
| | | | | | | 7.5 mL | | |
| | Citric acid and | 10 | AB-10 | 0 | 0 | — | 1 | 80 |
| | sorbitol wt % | 11 | AB-11 | 10 | 3 | — | 1 | 80 |
| | | 12 | AB-12 | 40 | 13 | — | 1 | 80 |

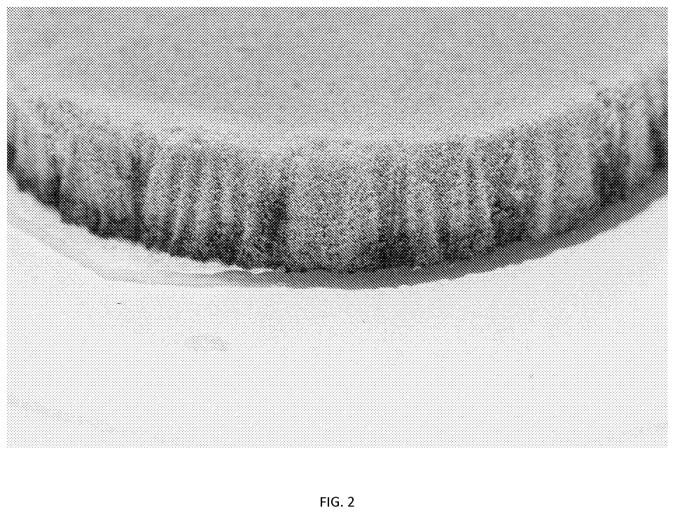

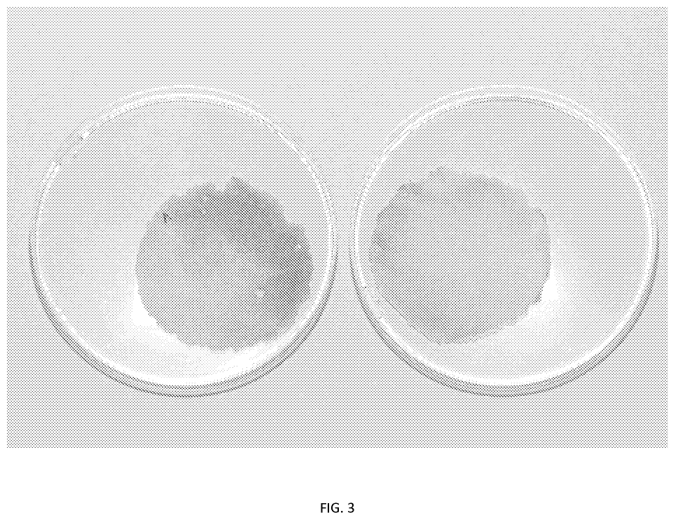

Initially, the treatment transformed the filtered cells and cell aggregates into a puree-like mixture. The dry, soft, and porous structures ultimately resulting from subsequent freeze-drying of the treated plant cells were termed “plant cell patties” (FIG. 2).

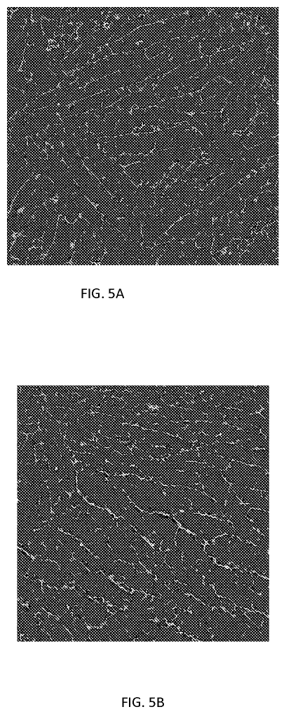

Most of the samples featured hollow and well-organized channels after freeze drying (FIG. 5) except those samples prepared without incubation, those treated with ammonium hydroxide, and the arctic bramble sample incubated at 20° C. For the rowan patties, the porosity value was 88-95%, the average pore size was 77-167 μm, and the wall thickness was 12-15 μm. For the arctic bramble patties, the porosity value was 85-95%, the average pore size was 72-214 μm, and the wall thickness was 11-15 μm (Table 2). Prolonged incubation reduced the porosity of rowan cell patties from 93% to 88% and the average pore size from 125 to 77 μm. Similarly, it reduced the porosity of artic bramble cell patties from 92% to 85% and the average pore size from 107 to 72 μm. This resulted in a more compact and closed structure compared to the control samples (Table 2). No incubation or incubation at 20° C. produced less-organized, heterogeneous structures with thinner walls (12 and 8 μm for rowan and arctic bramble, respectively, without incubation, and 12 and 13 μm, respectively, for incubation at 20° C.). The addition of ammonium hydroxide increased the pore size in rowan patties to 167 μm (low dose) and 140 μm (high dose) and in arctic bramble patties to 215 μm (low dose) and 167 μm (high dose), as well as broadening the pore size distribution (FIG. S2) and generating an unorganized, fragmented structure (FIG. S3). Ammonium hydroxide had a limited effect on wall thickness (15 and 14 μm in rowan and article cell patties, respectively, at the low dose, and 13 μm in both patties at the high dose).

Sodium hydroxide additions, incubation at 140° C. or different citric acid and sorbitol additions had no significant effect on pore size and its distribution.

### Mechanical Properties and Sensory Profiles of Plant Cell Patties

All samples were equilibrated to 43% relative humidity to avoid textural differences caused by variations in moisture content. Patties formed by the addition of citric acid and sorbitol alone featured a sticky upper layer, whereas those also incorporating sodium hydroxide had dry surfaces, and those containing a high dose of ammonium hydroxide featured smooth and shiny surfaces. Visual inspection also revealed differences caused by varying the amount of citric acid and sorbitol, with lower concentrations associated with crumbly, less cohesive patties.

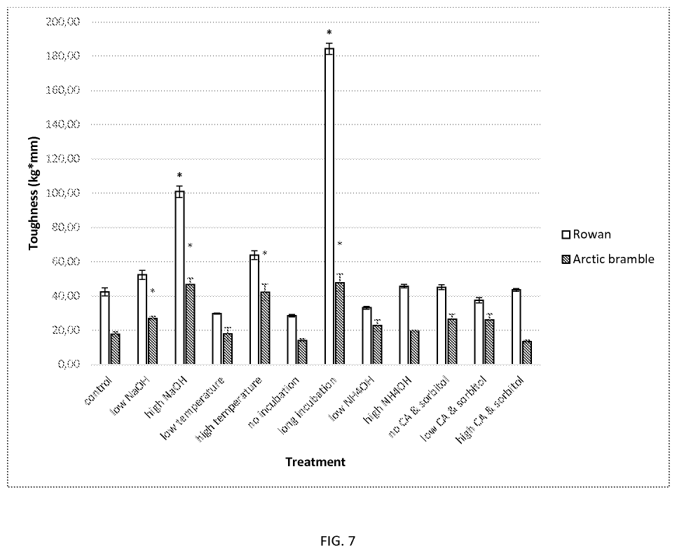

Toughness values extracted from force-deformation curve are presented in FIG. 7. For rowan cell patties, high levels of NaOH addition and long incubation time significantly increased the toughness values compared to control sample. For arctic bramble, NaOH addition gave statistically higher toughness values compared to control sample regardless of addition level. Yield point forces, indicating the elastic limit of the sample, were detected only for rowan samples with high NaOH addition 10.3 kg (n=4), high incubation temperature 9.2 kg (n=3), long incubation time 26.5 kg (n=2) and high NH₄OH addition 3.7 kg (n=3). With arctic bramble yield point forces were detected for high NaOH addition 4.4 kg (n=4), high incubation temperature 3.7 kg (n=4) and for long incubation time 13.2 kg (n=2).

Fresh arctic bramble cells were light yellow in color with a bright green shade but freeze-drying yielded patties that were lighter in color than fresh cells. The upper surface of the patty, which was in contact with air, was darker and more variable than the homogenous profile of the inner structure. Although short-term storage at room temperature and 43% relative humidity resulted in slightly bleached samples, prolonged storage (2 months) resulted in browning, especially in samples with high levels of added hydroxide, whereas no dramatic change in color was observed for the other samples.

Total color differences compared to control samples varied between ΔE=3−18 and ΔE=3−14 for the rowan and arctic bramble patties, respectively. For rowan samples, the greatest color differences were observed in patties containing high doses of sodium hydroxide (ΔE=18) and ammonium hydroxide (ΔE=11). For arctic bramble patties, the greatest color differences were observed in patties containing high doses of ammonium hydroxide (ΔE=14) and those lacking citric acid and sorbitol (ΔE=9).

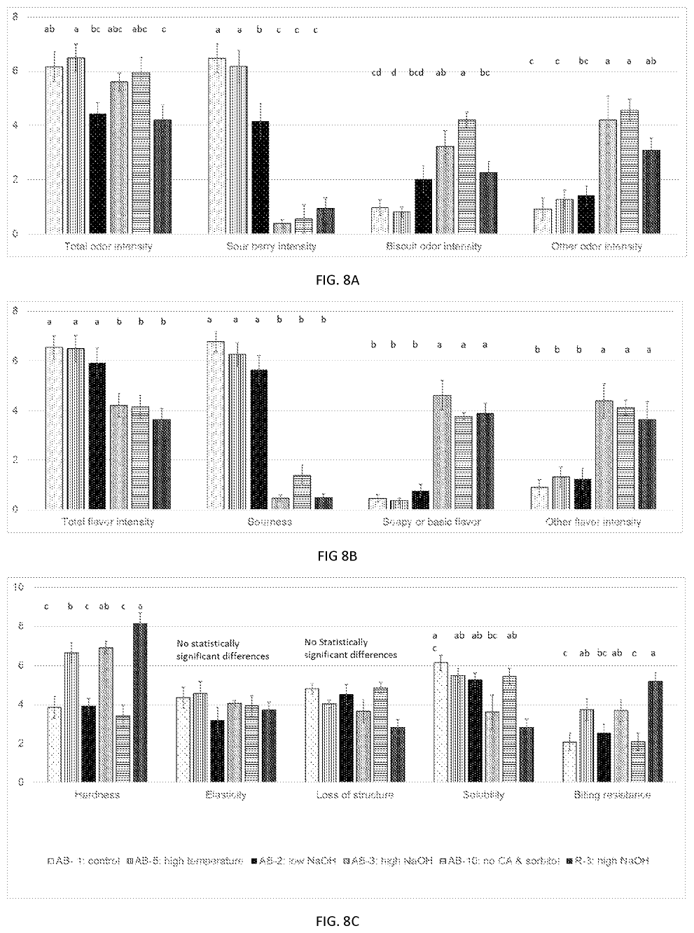

All samples selected for sensory evaluation passed the microbiological quality tests, with CFU counts below the threshold of 10 CFU/g or 1 CFU/mL. In the sensory profiling experiment, six samples were evaluated for differences in 13 different sensory attributes relating to odour, flavour and structure. There were statistically significant differences between samples in all attributes except elasticity (FIG. 8). The main treatments that affected the sensory profiles were the addition of sodium hydroxide and citric acid, which divided the samples into two broad groups: (1) samples containing citric acid but low or zero sodium hydroxide, and (2) samples containing no citric acid plus samples containing high-dose sodium hydroxide. Group (1) samples were characterized by an intense sour berry odor and sour taste and were highly soluble. In group (2), the lack of citric acid or particularly the high dose of sodium hydroxide caused an overall milder flavor, but more soapiness, hardness and biting resistance, and lower solubility. The perceived value of the loss of structure attribute in group (2) samples was comparable to that of extruded corn snacks, indicating that samples with added sodium hydroxide remained cohesive even after rehydration with saliva in the mouth. A higher incubation temperature increased the hardness of the samples but had a negligible impact on the flavor.

### REFERENCES

Hildebrand, T. & Ruegsegger, P. A new method for the model-independent assessment of thickness in three-dimensional images. J. Microsc. 185, 67-75 (1997).
Häkkinen, S. T. et al. Plant cell cultures as food—aspects of sustainability and safety. Plant Cell Rep. 39, 1655-1668 (2020).
Murashige, T., & Skoog, F. (1962). A revised medium for rapid growth and bioassays with tobacco tissue cultures. Physiologia Plantarum, 15, 473-497 Nordlund, E. et al. Plant cells as food—A concept taking shape. Food Res. Int. 107, 297-305 (2018).
Sozer, N., Dogan, H. & Kokini, J. L. Textural properties and their correlation to cell structure in porous food materials. J. Agric. Food Chem. 59, 1498-1507 (2011).
Suvanto, J. et al. Variability in the production of tannins and other polyphenols in cell cultures of 12 Nordic plant species. Planta 246, 227-241 (2017).
WO 2010/059725

1. A process for converting biomass derived from a plant cell suspension culture into a three-dimensional plant biomass structure, characterized in that the process comprises the steps of:
providing a plant cell suspension culture comprising plant cells and culture medium;
growing the plant cells in the culture medium;
separating the plant cells from the culture medium by removing the culture medium to obtain plant cell mass;
treating the plant cell mass with citric acid by mixing the plant cell mass and citric acid;
formulating the plant cell mass into a three-dimensional shape;
incubating the plant cell mass at a temperature of 15 to 250° C. for 30 minutes to 24 hours; and
dehydrating the plant cell mass to obtain a three-dimensional plant biomass structure.

2. The process according to claim 1, characterized in that the plant cells are separated from the culture medium by filtration, centrifugation, sedimentation, or precipitation, or a combination thereof.

3. The process according to claim 1, characterized in that a humidifying agent is added to the plant cell mass.

4. The process according to claim 1, characterized in that water is added to the plant cell mass.

5. The process according to claim 1, characterized in that the plant cell mass is treated with a base to adjust pH of the plant cell mass to a desired level.

6. The process according to claim 5, characterized in that the base is selected from the group consisting of sodium hydroxide, sodium bicarbonate, ammonium bicarbonate, ammonia, potassium bicarbonate, calcium hydroxide, calcium carbonate, trisodium phosphate, sodium benzoate, and ammonium hydroxide.

7. The process according to claim 1, characterized in that the plant cell mass is dehydrated by freeze drying.

8. A three-dimensional plant biomass structure obtained by the process according to claim 1 characterized in that the structure has at least one property selected from the group consisting of: a porosity value between 85-95%, toughness level between 13-185 kg*mm, and hardness level between 6-108 kg.

9. A three-dimensional plant biomass structure according to claim 1, characterized in that the structure has a porosity value between 85-95%, and at least one property selected from the group consisting of: toughness level between 13-185 kg*mm and hardness level between 6-108 kg

10. A three-dimensional plant biomass structure according to claim 8, characterized in that the structure has a porosity value between 85-95%, toughness level between 13-185 kg*mm, and hardness level between 6-108 kg.

11. A food product comprising the three-dimensional plant biomass structure according to claim 8, characterized in that the food product comprises one or more further ingredients selected from the group consisting of proteins, fats, polysaccharides, colourings, flavourings, preservatives, salt, antioxidants, and sugars.

12. The food product according to claim 11, characterized in that the food product is selected from the group consisting of a bar, snack, snack bar, muesli, cracker, chip, topping, garnish, and decoration.

13. Use of a three-dimensional plant biomass structure according to claim 8 as an ingredient in a food product.

14. Use according to claim 13, characterized in that the three-dimensional plant biomass structure is used as a sole ingredient.

15. Use according to claim 13, characterized in that the three-dimensional plant biomass structure is used in addition to another ingredient or with another ingredient.
